(19) United States
(12) Patent Application Publication
(10) Pub. No.: US 2023/0212089 A1
(43) Pub. Date: Jul. 6, 2023

(54) ORGANOMINERAL FERTILIZER

(71) Applicant: Petróleo Brasileiro S.A. - Petrobras, Rio de Janeiro (BR)

(72) Inventors: Leonardo Brantes Bacellar Mendes, Rio de Janeiro (BR); Heraldo Namorato De Souza, Rio de Janeiro (BR); Rafael Richard Joao, Rio de Janeiro (BR); Ronaldo Bernardo Da Silva, Rio de Janeiro (BR)

(21) Appl. No.: 18/087,453

(22) Filed: Dec. 22, 2022

(30) Foreign Application Priority Data
Dec. 30, 2021 (BR) 10 2021 026904 9

(51) Int. Cl. C05G 1/00; C05F 11/08; C05D 9/00

(52) U.S. Cl. CPC C05G 1/00; C05F 11/08; C05D 9/00

(57) ABSTRACT

The present invention addresses to the production of an organomineral fertilizer consisting of a mineral matrix, drilling gravel and an organic matrix, algae biomass. Algae are cultivated using drilling gravel suspended in the culture medium together with their ability to grow by absorbing CO₂, and, in the case of cyanobacteria, they fix atmospheric nitrogen in their biomass. The gravel provides a set of additional nutrients present in its composition to the conventional nutritional chemical compounds for algae. In this way, the gravel can be mixed with the culture medium in the cultivation of algae, generating the organic matrix to be formulated and commercialized in the national and international agricultural market as an organomineral fertilizer, and, in the case originating from the organic matrix of cyanobacteria, there is the enrichment with nitrogen obtained biologically, without the high energy expenditure typical of traditional synthetic nitrogen fertilizers. In this way, the present invention enables the disposal with a minimum of environmental impact and constitutes a satisfactory solution for the disposal of gravel for oil E&P.

### FIELD OF THE INVENTION

The present invention addresses to an organomineral fertilizer formulated with a mineral matrix based on the residue of drilling gravel generated in oil exploration and production (E&P) activities associated with an organic algal matrix produced by cyanobacteria, microalgae and macroalgae in media of cultivation, aiming at valuing and making viable an environmentally sustainable alternative for waste disposal and in the production and sale of organomineral fertilizer through the use of drilling gravel from E&P activities.

### DESCRIPTION OF THE STATE OF THE ART

Algae are found from aquatic environments to arid terrestrial environments. They are sources of organic matter for soils, as their photosynthetic rates are about 10-50 times faster than higher plants. Their biomolecules and recalcitrant cells contribute to the accumulation of carbon in the soil. Algae present in the soil have the property of increasing the resilience of cultivated plants against abiotic stress factors, such as drought and saline and sadistic soils. The market for organomineral fertilizers is currently expanding in Brazil and in the world and its demand for agriculture, farming and forestry is expected to grow even more.

The advantages of these fertilizers are the increase in agricultural productivity and the positive environmental impacts, as they promote the fixation of carbon in the soil (each 100 kg of organomineral fertilizer promotes the fixation of 33 kg of carbon in the soil) without depending on the oil industry and gas for its production, which can be advantageously associated (competitive advantage) with mineral compounds producing organomineral fertilizer. The raw material sources of mineral matrix for organomineral fertilizers can come from solid waste generated by E&P activities. Waste produced in E&P activities constitutes an environmental liability that implies high costs due to the need for treatment and disposal according to existing environmental regulations and constant subjection to the risk of regulatory infractions.

The amount of drilling gravel generated by the offshore E&P and landed for treatment and disposal on land is around 50 ton/year (year 2020, Petrobras data). The remainder of the gravel produced is discarded at sea, as long as it is not environmentally unsuitable. With IBAMA's normative instruction No. 1/2018, currently suspended, a rapid reduction in the disposal of drilling gravel at sea was foreseen, up to the total prohibition of disposal, passing through an intermediate phase with the collection of gravel from the reservoir phase of the wells. In the first phase, all the gravel from the reservoir phase should be sent to land (17,000 ton/year, estimates for Petrobras) and, in the second phase, comprises the implementation of zero disposal, where all the gravel generated must be collected (70,000 ton/year, estimate for Petrobras). These are significant amounts, without a viable technological alternative for treatment and with a possible current destination for disposal in industrial landfills, with major impacts on soils and aquifers, due to the large amount of salt present with the potential to cause salinization and sodization in these environments.

When drilling oil wells, large volumes of drilling gravel are produced, which require treatment and disposal for proper disposal in the environment or reuse or recycling. The composition of drilling gravel depends on the type of drilling fluid used and the mineralogical composition of the rocks drilled during well construction. Improper treatment and disposal of gravel can trigger several environmental impacts, such as salinization, sodization and contamination of soil and aquifers due to high levels of salts and sodium and possible high levels of oil hydrocarbons.

Cyanobacteria, microalgae and macroalgae, referred to here as algae, have mechanisms used in soils to tolerate salinity, sodicity and the contamination of oil hydrocarbons, from which, directly or indirectly, cultivated plants also benefit because they will also be present these mechanisms in the organic matrix originating from algae that participate in the formulation of organomineral fertilizers used in the fertilization programs of these cultivated plants, neutralizing or minimizing the possible negative effects of the gravel on the soil and plants, enabling its destination with minimal environmental impact and constituting a satisfactory environmental solution for the disposal of gravel for oil E&P.

Document REIS, F. P. (2021) “Reuso de cascalho de perfuracão de pocos de petróleo para producão de adubo organomineral: aspectos ecotoxicológicos”, Scientific and Technological Initiation Seminar Federal University of Santa Catarina—Center for Rural Sciences Forestry Engineering, discloses the application of reuse of gravel from oil drilling as fertilizers and its ecotoxicological aspects. The study analyzes the feasibility of reusing this by-product as an organomineral fertilizer, in which gravel concentrations (1 to 15%) were evaluated in tropical artificial soil, where ecotoxicological tests were used with soil invertebrates and plant species, following ABNT or ISO standards with some adaptations.

LIMA, L. S. (2013) “Influência do tratamento de lavagem de resíduos de perfuração de pocos de petróleo no desenvolvimento de plantas de arroz (Oryza sativa)”, 95f., Dissertation (Master's degree)—Federal Rural University of Rio de Janeiro, Postgraduate Course in Agronomy—Soil Science, focuses on clarifying that drilling gravel washing can be positive for agricultural crops (rice) without stating or clarifying or proving at any time that there may even be microalgae cultivation with high growth that can generate organomineral compost to be used as an agricultural input. In addition, there is no mention of microalgae nor of the use of saline and sodium drilling gravel as a component of the culture medium for nutrition and factors for growth and development of algae.

PEREIRA, G. H. M. (2016) “Cultivo da microalga Nannochloropsis” of Graduation in Aquaculture Engineering, Federal University of Santa Catarina, is based on clarifying that Nannochloropsis can be cultivated in batch mode fed by two different culture media generating growth comparisons—conventional approach that at no time even mentions the existence of drilling gravel or demonstrates innovation in the combination of drilling waste and the associated development of Nannochloropsis or other microalgae such as Desmodesmus.

Document CA2874890A1A discloses a bioavailable mineral fertilizer composition comprising a full spectrum of essential minerals and organic carbon nutrients for soil and plant health incorporated into a composite mixing medium chosen from the group comprising liquid, powder, granular. However, this reference does not show results in which microalgae can be developed “in vivo” in direct contact with the drilling gravel with the generation of an organomineral fertilizer that comes directly associated with the biotechnological process. Further, the reference to algae is general and there is no mention of the use of saline and sodic drilling gravel as a component of the culture medium for nutrition and factors for growth and development of algae.

In view of this, no document of the state of the art discloses the use of drilling gravel from oil wells associated with cyanobacteria, microalgae or macroalgae to obtain organomineral fertilizers such as that of the present invention.

Thus, the present invention proposes an organomineral fertilizer product formulated with a mineral matrix based on the drilling gravel residue generated in E&P activities associated with an organic matrix consisting of algal biomass produced by the cultivation of cyanobacteria, microalgae and macroalgae in cultivation media with the predominant use of drilling gravel as the main source of nutrients for the nutrition of these algae.

The resulting organomineral fertilizer has mechanisms that stimulate the tolerance of fertilized plants to salinity, sodicity and possible contamination of oil hydrocarbons present in the drilling gravel used in the formulation of this organomineral fertilizer, where the active mechanisms are: stimulation of the production of extracellular polysaccharides by plants to buffer the salt effects present in the rhizosphere; release of organic acids through the microbial decomposition of organic matter from the algal biomass that react with the calcium carbonate in the drilling gravel, releasing the calcium nutrient for the plants and forming carbonic acid that would stimulate the development and proliferation of algae in the soil; synthesis and accumulation of osmoregulatory compounds, such as sugars and quaternary amines, raising the osmotic tension of plant roots and consequently increasing the absorption of water and nutrients; maintain low levels of internal sodium in plants; chelators present in algal biomass will decrease sodium toxicity to plants and excess calcium in the soil solution; among other mechanisms.

Thus, the present invention presents advantages in fulfilling the sustainability commitments established by reducing the generation of hazardous and non-hazardous waste, drilling gravel, by its reuse or reuse as an organomineral fertilizer.

Thus, the present invention allows the use of environmental liabilities consisting of solid waste from drilling activities coupled with biofixation of CO₂ and, in the case of cyanobacteria, fix atmospheric nitrogen in their biomass enriched with nitrogen obtained in a way biological, without the high energy expenditure typical of traditional synthetic nitrogen fertilizers, to obtain organomineral fertilizer, in such a way that there is the addition of value to an environmental liability generated by oil and gas E&P activities.

### BRIEF DESCRIPTION OF THE INVENTION

The present invention addresses to an organomineral fertilizer formulated with a mineral matrix based on the drilling gravel residue generated in E&P activities associated with an organic matrix consisting of algal biomass produced by the cultivation of cyanobacteria, microalgae and macroalgae in cultivation media with the predominant use of drilling gravel as the main source of nutrients for the nutrition of these algae.

The resulting organomineral fertilizer has mechanisms that will stimulate the tolerance of fertilized plants to salinity, sodicity and possible contamination of oil hydrocarbons present in the drilling gravel used as a mineral matrix in the formulation of this organomineral fertilizer.

The invention applies to the valuation and feasibility of an environmentally sustainable alternative for waste disposal and the marketing of an innovative product through the use of drilling gravel residue from E&P activities (for example, pre-salt of the Santos basin) obtaining an organomineral fertilizer, as part of a transition to a low-carbon economy and with added value in the national and international agricultural input market.

### BRIEF DESCRIPTION OF THE DRAWINGS

The present invention will be described in more detail below, with reference to the attached figures which, in a schematic way and not limiting the inventive scope, represent examples of its realization. The drawings have:

FIG. 1 illustrating an organomineral fertilizer production process according to the present invention: solar radiation (1) falls on algal biomass production cultivation tanks (6), where cyanobacteria, microalgae or macroalgae inoculants (5) containing water and culture media (3), adding drilling gravel (2) and CO₂ (4). Algae biomass (7) is produced and collected to be used as an organomineral fertilizer (8);

FIG. 2 illustrating the beginning of the experiment, after inoculation with Nannochloropsis sp. (increase of 1000×), where (P) is the reactor with drilling gravel, and (C) the control reactor;

FIG. 3 illustrating Nannochloropsis sp. after 24 hours from the beginning of the experiment (increase of 1000×), where (P) is the reactor with drilling gravel, and (C) the control reactor;

FIG. 4 illustrating Nannochloropsis sp. after 48 hours from the beginning of the experiment (increase of 1000×); where (P) is the reactor with drilling gravel, and (C) the control reactor;

FIG. 5 illustrating the general aspect of the cultures throughout the experiment with agitation and injection of CO₂, wherein there are the denominated sample with addition of gravel from the pre-salt (P) and the denominated control sample (C);

FIG. 6 illustrating the general appearance of the cultures seven days after the end of the experiment, wherein there are the denominated sample with the addition of pre-salt gravel (P) and the denominated control sample (C).

### DETAILED DESCRIPTION OF THE INVENTION

The organomineral fertilizer according to the present invention comprises drilling gravel from the oil and gas industry (onshore or offshore), carbon dioxide, cyanobacterial culture or microalgae or macroalgae and culture medium.

To obtain the organomineral fertilizer, it is necessary to add drilling gravel from the oil and gas industry to the culture medium. Next, the inoculation of the strain (cyanobacteria, microalgae or macroalgae) is carried out. CO₂ is added to the system to control the pH. The strain used grows by absorbing the CO₂ and using the nutrients provided in the added mixture (drilling gravel+culture medium). The organic matrix generated after cultivation together with the mineral matrix from the drilling gravel will be the final product that will be marketed as an organomineral fertilizer (FIG. 1).

More specifically, the organic matrix is produced from the cultivation of microalgae, cyanobacteria and macroalgae with the addition of drilling gravel from any origin, whether from the pre-salt offshore, post-salt offshore, onshore, among other origins associated with the offshore and onshore environment, in concentrations based on mass/mass (kg/kg) or mass/volume (kg/L) units ranging from 0.01% to 95%, ideally 0.1%, in a culture medium or a combination of culture media. The inoculation of Nannochloropsis sp. in concentrations ranging from 1 mg/L to 1 g/L, with an ideal of 50 mg/L, in tanks or other types of transparent containers subjected to natural or artificial sunlight with a light intensity greater than 60 klux with an ideal of 120 klux, with a photoperiod of 8 to 14 hours, ideally of 12 hours. The supply of CO₂ has no limitation of purity (ideally with 99% purity), as long as there is no presence of other gases in concentrations that are toxic to microalgae, cyanobacteria and macroalgae. The pH was maintained between 6 and 7, ideally with a pH of 6.5 over 36 hours to 6 days of growth, ideally three days. Salinity, measured with reference to sodium chloride, between 0.001 to 150 g/L, ideally 35 g/L. The temperature between 24 to 48° C., ideally at 36° C.

The organomineral fertilizer can be applied via soil, fertigation, hydroponics, via foliar and via plant seeds or other application form not listed here. Its physical nature can be granular comprising a solid product made up of particles in which each granule contains all the nutritional chemical elements for plants declared or guaranteed in the product. Its physical nature can also be fluid, comprising a liquid, pasty or gel product, in solution or suspension. It is understood as:
1—solid: physical nature of the product consisting of solid particles or fractions;
2—solution: specification of the physical nature of a fluid product without solid particles;
3—suspension: specification of the physical nature of a fluid product with solid particles dispersed in a fluid medium.

Nutrient chemical elements are expressed as contents in the units indicated by the relevant legislation as primary macronutrients, secondary macronutrients, micronutrients and beneficial elements of organomineral fertilizers as follows:
a—primary macronutrients: Nitrogen (N), Phosphorus (P) and Potassium (K);
b—secondary macronutrients: Calcium (Ca), Magnesium (Mg) and Sulfur (S);
c—micronutrients: Boron (B), Chlorine (Cl), Cobalt (Co), Copper (Cu), Iron (Fe), Manganese (Mn), Molybdenum (Mo), Nickel (Ni), Selenium (Se), Silicon (Si) and Zinc (Zn), among others to be discovered in the future by scientific research;
d—beneficial elements: Sodium (Na), Silicon (Si), among others to be discovered in the future by scientific research.

The organomineral fertilizer comprises the following components:
mineral matrix, chosen from the drilling gravel;
organic matrix consisting of algal biomass produced by cultivating cyanobacteria, microalgae or macroalgae in cultivating media based on drilling gravel.

The combination of mineral and organic matrix will be formulated in proportions that meet the criteria and parameters described below.

The solid or fluid organomineral fertilizers for application to the soil or fertigation must comply with the following:
1—organic carbon: minimum of 8% (eight percent) for solid product and 3% (three percent) for fluid product;
2—humidity: maximum of 20% (twenty percent) for solid product and greater than 20% (twenty percent) for fluid product;
3—cation exchange capacity (CEC): minimum of 80 (eighty) mmol_{c}/kg for solid product and no limitation for fluid product;
4—it may contain all or part of it, in various ratios, proportions, formulations, minimum and maximum limits of the primary and secondary macronutrients, micronutrients and guaranteed or declared beneficial elements of the product.

The solid or fluid organomineral fertilizers for foliar application, via hydroponics and via seeds must comply with the following:
1—The solid product must be soluble in water;
2—There can be added chelating agents, complexing agents or authorized additives, according to the relevant legislation.

### EXAMPLES

The following examples are presented in order to more fully illustrate the nature of the present invention and the way to practice the same, without, however, being considered as limiting its content.

### Example 1

### Dynamic Experiment under Sunlight with Agitation and CO₂ Injection—Methodology Used.

1 g of ground gravel (containing Al, Fe, Ca, Mg, Cu, Mn, Zn, Ni, Cr, P, K and Na) from drilling activities in the pre-salt layer (Pre-salt well: 9-MLL-83-RJS) to a mixture of F2 culture medium (800 ml) and BG11 medium (200 ml). This mixture was inoculated with Nannochloropsis sp. in low concentration (about 50 mg/l) in a transparent bottle exposed to natural sunlight (light intensity of 120 klux).

At the same time, another inoculation was carried out with a similar concentration and under the same lighting condition (120 klux) with a mixture of F2 medium and BG11 medium without drilling gravel, maintained as a control over time. CO₂ (99% purity) was supplied to the bottles through a solenoid valve triggered by a pH controller with a glass electrode. The pH was maintained at 6.5 over three days of growth (72 hours). Abiotic parameters observed throughout the experiment:
Salinity: 35 g/L;
Light intensity: 120 klux (photoperiod of 12 hours/day);
Temperature: 36° C.;
CO₂ injection: Performed;
pH: 6.5 for sample with gravel;
pH: 6.5 for control sample.

CO₂ (99% purity) was supplied to the bottles through a solenoid valve triggered by a pH controller with a glass electrode. The pH was maintained at 6.5 over three days of growth (72 h).

### Biotic Parameters Observed Throughout the Experiment:

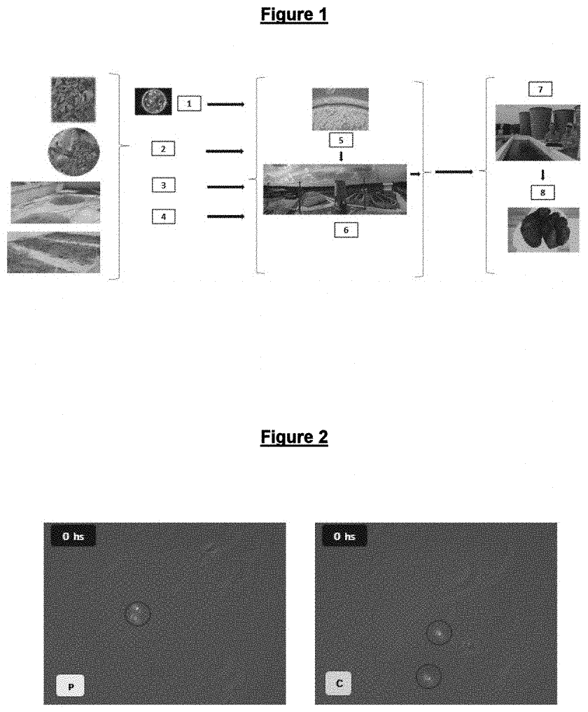

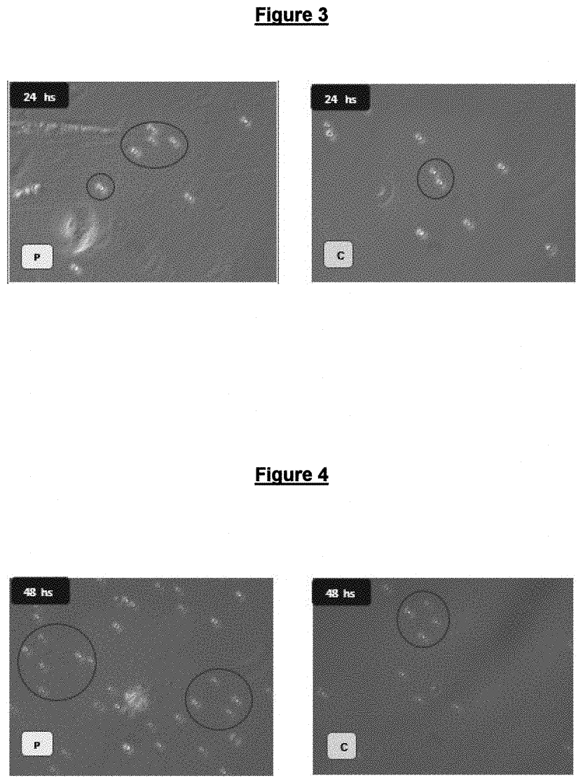

Daily observations were carried out under an optical microscope (FIGS. 2, 3 and 4) and also macroscopic observations (FIG. 5) that attested to the presence of healthy microalgae (Nannochloropsis sp.) growing in the bottle containing gravel (P) and in the control bottle (C).

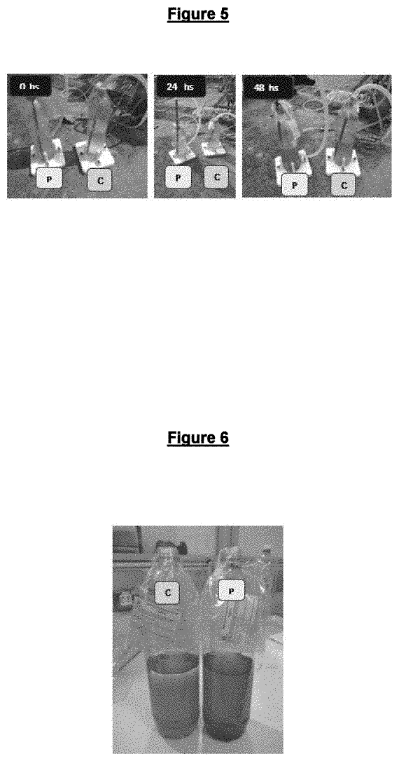

After the end of the experiment, the bottles containing the cultures were kept without agitation for a week, having shown high stability in the microalgae of the control culture and in the microalgae that grew in the medium containing gravel from the pre-salt (FIG. 6).

At the end of the experiment, it was found that there was excellent growth of Nannochloropsis sp. with CO₂ capture and using gravel from the pre-salt at a concentration of 1 g/l in the mixture of F2 and BG11 medium.

In addition, high stability was observed in the microalgae of the control culture and in the microalgae that grew in the medium containing gravel from the pre-salt layer, indicating a high potential for storage, if it is marketed as an organomineral fertilizer (FIG. 6). There was no visible difference between the control sample and the one containing gravel with regard to the growth obtained and the quality of the cells in both conditions. Nannochloropsis sp. demonstrated affinity with culture medium containing this type of solid residue, which represents an innovative result in relation to the international literature.

It should be noted that, although the present invention has been described in relation to the attached drawings, it may undergo modifications and adaptations by technicians skilled on the subject, depending on the specific situation, but provided that within the inventive scope defined herein.

1. AN ORGANOMINERAL FERTILIZER, characterized in that it comprises:
a mineral matrix, chosen from the drilling gravel;
an organic matrix consisting of algal biomass produced by cultivating cyanobacteria, microalgae or macroalgae in culture media based on drilling gravel.

2. THE ORGANOMINERAL FERTILIZER according to claim 1, characterized in that it is for application via soil, fertigation, hydroponics, via foliar application and via plant seeds.

3. THE ORGANOMINERAL FERTILIZER according to claim 1, characterized in that it is a solid, liquid, pasty or gel product, in solution or suspension.

4. THE ORGANOMINERAL FERTILIZER according to claim 1, characterized in that the combination of the mineral and organic matrix presents the following proportions:
1—organic carbon: minimum of 8% for solid product and 3% for fluid product;
2—humidity: a maximum of 20% for a solid product and greater than 20% for a fluid product;
3—cation exchange capacity (CEC): minimum of 80 mmol_{c}/kg for solid product and no limitation for fluid product;
4—contain all or part, in various ratios, proportions, formulations, minimum and maximum limits of primary and secondary macronutrients, micronutrients and guaranteed or declared beneficial elements of the product.

5. THE ORGANOMINERAL FERTILIZER according to claim 4, characterized in that, additionally, the solid product is soluble in water; and can add chelating agents, complexing agents or additives.

6. THE ORGANOMINERAL FERTILIZER according to claim 1, characterized in that the organic matrix is produced from the cultivation of microalgae, cyanobacteria and macroalgae with the addition of drilling gravel in concentrations ranging from 0.01% to 95%, preferably of 0.1%, in culture medium or combination of culture media; wherein the inoculation of Nannochloropsis sp. in concentrations ranging from 1 mg/L to 1 g/L, preferably 50 mg/L, in tanks or other types of transparent containers subjected to natural or artificial sunlight with a light intensity greater than 60 klux with an ideal of 120 klux, with photoperiod from 8 to 14 hours, preferably 12 hours; wherein the CO2 supply is without limitation of purity, preferably 99% purity; wherein the pH is maintained between 6 and 7, preferably pH 6.5 over 36 hours to 6 days of growth, preferably three days; wherein the salinity is measured with reference to sodium chloride, between 0.001 to 150 g/L, preferably 35 g/L; wherein the temperature is between 24 to 48° C., preferably 36° C.
